(19) United States
(12) Patent Application Publication
(10) Pub. No.: US 2021/0308075 A1
(43) Pub. Date: Oct. 7, 2021

(54) PREVENTION AND TREATMENT OF VIRIAL INFECTIONS

(71) Applicant: Joseph Habboushe, New York, NY (US)

(72) Inventor: Joseph Habboushe, New York, NY (US)

(21) Appl. No.: 17/224,530

(22) Filed: Apr. 7, 2021

Related U.S. Application Data
(60) Provisional application No. 63/006,257, filed on Apr. 7, 2020.

(51) Int. Cl. A61K 31/135; A61K 31/192

(52) U.S. Cl. CPC A61K 31/135; A61K 31/192

(57) ABSTRACT

The present disclosure relates generally to compositions and methods for treating infections by highly virulent viruses, such as SARS-CoV-2, SARS-CoV, the H5N1 influenza A virus, and the H7N9 influenza A virus. The method can include administration of aspirin or ketamine, or a combination thereof.

### CROSS REFERENCE TO RELATED APPLICATIONS

This application claims the benefit under 35 U.S.C. § 119(e) of U.S. Provisional Application Ser. No. 63/006,257, filed Apr. 7, 2020, the content of which is incorporated by reference in its entirety into the present disclosure.

### BACKGROUND

Severe acute respiratory syndrome coronavirus 2 (SARS-CoV-2), previously known by the provisional name 2019 novel coronavirus (2019-nCoV), is a positive-sense single-stranded RNA virus. It is contagious in humans and is the cause of the ongoing pandemic of coronavirus disease 2019 (COVID-19) that has been designated a Public Health Emergency of International Concern by the World Health Organization (WHO).

SARS-CoV-2 has close genetic similarity to bat coronaviruses, from which it likely originated. From a taxonomic perspective, SARS-CoV-2 is classified as a strain of the species severe acute respiratory syndrome-related coronavirus (SARSr-CoV).

Each SARS-CoV-2 virion is approximately 50-200 nanometres in diameter. Like other coronaviruses, SARS-CoV-2 has four structural proteins, known as the S (spike), E (envelope), M (membrane), and N (nucleocapsid) proteins; the N protein holds the RNA genome, and the S, E, and M proteins together create the viral envelope. The spike protein, which has been imaged at the atomic level using cryogenic electron microscopy, is the protein responsible for allowing the virus to attach to the membrane of a host cell.

Protein modeling experiments on the spike protein of the virus soon suggested that SARS-CoV-2 has sufficient affinity to the angiotensin converting enzyme 2 (ACE2) receptors of human cells to use them as a mechanism of cell entry. Studies have shown that SARS-CoV-2 has a higher affinity to human ACE2 than the original SARS virus strain. SARS-CoV-2 may also use basigin to gain cell entry.

Coronavirus disease 2019 (COVID-19) refers to the disease caused by infection with SARS-CoV-2. Common symptoms include fever, cough, and shortness of breath. Muscle pain, sputum production and sore throat are less common. While the majority of cases result in mild symptoms, some progress to severe pneumonia and multi-organ failure.

Research into potential treatments started in January 2020, and several antiviral drugs are in clinical trials. Although new medications may take until 2021 to develop, several of the medications being tested are already approved for other uses, or are already in advanced testing.

### SUMMARY

The present disclosure, in one embodiment, provides compositions and methods for treating or preventing infections by highly virulent viruses, such as SARS-CoV-2, severe acute respiratory syndrome coronavirus (SARS-CoV), which is the strain of virus that caused the severe acute respiratory syndrome (SARS) outbreak in 2003 in Asia, the H5N1 influenza A virus, and the H7N9 influenza virus.

The methods, in some embodiments, entail administering a patient having, or at risk of developing, the infection an effective amount of ketamine. In some embodiments, the ketamine is orally administered. In some embodiments, a first portion of the ketamine disintegrates or dissolves intraorally, and a second portion of the ketamine is ingested and released in the gastrointestinal track of the patient.

In some embodiments, the methods further entail administering to the patient an effective amount of aspirin. In some embodiments, a first portion of the aspirin disintegrates or dissolves intraorally, and a second portion of the aspirin is ingested and released in the gastrointestinal track of the patient.

Also provided, in one embodiment, is a method of treating or preventing the infection by a virus in a subject, comprising orally administering to the subject a first amount of aspirin and a second amount of aspirin, wherein the first amount disintegrates or dissolves intraorally providing rapid release of the aspirin of the first amount, and the second amount is ingested and released in the gastrointestinal track of the subject. In some embodiments, the second amount of aspirin is administered with ketamine. In some embodiments, the first amount of aspirin is administered with ketamine.

In some embodiments, the virus is a coronavirus. In some embodiments, the coronavirus is SARS-CoV-2 or SARS-CoV. In some embodiments, the virus is an influenza virus. In some embodiments, the influenza virus is the H5N1 influenza A virus or the H7N9 influenza A virus.

In some embodiments, the patient suffers from a neuropathy. Non-limiting examples of neuropathy include anosmia, cognitive or attention deficit, anxiety, depression, psychosis, seizure, suicidal behavior and combinations thereof.

### DETAILED DESCRIPTION

Unless defined otherwise, the terms used herein are intended to have their ordinary meaning in the art.

All numerical designations, e.g., pH, temperature, time, concentration, and weight, including ranges, are approximations that typically may be varied (+) or (−) by increments of 0.1, 1.0, 10.0, or 100.0 as appropriate. It is to be understood, although not always explicitly stated, that all numerical designations are preceded by the term “about”.

“About” will be understood by persons of ordinary skill in the art and will vary to some extent on the context in which the term is used. If there are uses of the term which are not clear to persons of ordinary skill in the art given the context in which it is used, “about” will mean up to plus or minus 10%, or 5%, or 2% or 1% or 0.5% of the particular term.

“Administering” or “administration of” a drug to a patient (and grammatical equivalents of this phrase) refers to direct administration, which may be administration to a patient by a medical professional or may be self-administration, and/or indirect administration, which may be the act of prescribing a drug. For example, a physician who instructs a patient to self-administer a drug and/or provides a patient with a prescription for a drug is administering the drug to the patient.

As used herein, “compressed” dosage form (e.g., “compressed portion”), refers to a dosage form comprising a compressed powder. For example, a compressed portion may be formed using a rotary tablet press or other similar machinery known to one of skill in the art.

As used here, “disintegrates or dissolves intraorally” refers to that a majority of a composition or a portion of a composition, such as a tablet or a capsule, breaks apart into smaller particles intraorally. The majority, in one aspect, means at least about 50%, or alternatively at about 60%, or 70%, or 80%, or 90%, or 95%, or 98%, or 99%.

As used herein, “bilayer” compressed dosage form (e.g., “bilayer tablet”) refers to a single compressed dosage form comprising two layers. A bilayer compressed dosage form can be made in a single compression step. Likewise, a “trilayer” compressed dosage form (e.g., “trilayer tablet”) refers to a single compressed dosage form comprising three layers.

As used herein, “wet granulation” refers to a process known in the pharmaceutical arts that involves forming granules by the addition of a liquid, such as purified water, alcohol, or a binder solution.

“Controlled release form” refers to a formulation in which the niacin is included within a matrix, which matrix can be either insoluble, soluble, or partly soluble. Controlled release matrix formulations of the insoluble type are also referred to as insoluble polymer matrices, swellable matrices, or lipid matrices depending on the components that make up the matrix. Controlled release matrix formulations of the soluble type are also referred to as hydrophilic colloid matrices, erodible matrices, or reservoir systems. Controlled release formulations of the present disclosure refer to formulations comprising an insoluble matrix, a soluble matrix or a combination of insoluble and soluble matrices in which the rate of release is slower than that of an uncoated non-matrix or immediate release formulations or uncoated normal release matrix formulations. Controlled release formulations can be coated with a control releasing coat to further slow the release of niacin from the controlled release matrix formulation. Such coated controlled release matrix formulations can exhibit modified-release, controlled-release, sustained-release, extended-release, prolonged-release, delayed-release, or combinations thereof, of niacin. Examples of controlled release forms of niacin include Slo-Niacin® available from Upsher Smith Laboratories, Inc. (Maple Grove, Minn.).

“Controlled release coat” refers to a functional coat which can, for example, include at least one pH independent or pH dependent (such as for example enteric or reverse enteric types) polymer, soluble or insoluble polymer, lipids or lipidic materials, or combinations thereof, which, when applied onto a formulation can slow (for example, when applied to an immediate release formulation or a normal release matrix formulation), further slow (for example when applied to a controlled release matrix formulation), or modify the rate of release of niacin.

“Excipient” refers to a pharmacologically inactive substance used with the active agents or drugs of a medication or a formulation. Excipients are also sometimes used to bulk up formulations that contain very potent active ingredients, to allow for convenient and accurate dosage. In addition to their use in the unit dose forms, excipients can be used in the manufacturing process to aid in the handling of the active substance concerned. Depending on the route of administration, and form of medication, different excipients may be used. Examples of an excipient includes, without limitation, one or more of the following: an additive, an anti-foaming agent, a binder, a chemical stabilizer, a coloring agent, a diluent, a disintegrating agent, an emulsifying agent, a filler, a flavoring agents, a glidant, a lubricant, a pH modifier, a plasticizer, a solubilizer, a swelling enhancer, a spheronization aid, a solubility enhancer, or a suspending agent.

“Immediate release formulation” refers to a formulation from which the drug is released without any substantial delay and substantially at once.

“Patient” or “subject” refers to mammals, including humans and animals, such as simians, cattle, horses, dogs, cats, and rodents having the need to take niacin.

“Pharmaceutically acceptable salt” refers to pharmaceutically acceptable salts derived from a variety of organic and inorganic counter ions well known in the art that include, by way of example only, sodium, potassium, calcium, magnesium, ammonium, and tetraalkylammonium, and when the molecule contains a basic functionality, salts of organic or inorganic acids, such as hydrochloride, hydrobromide, tartrate, mesylate, acetate, maleate, and oxalate. Suitable salts include those described in P. Heinrich Stahl, Camille G. Wermuth (Eds.), Handbook of Pharmaceutical Salts Properties, Selection, and Use, 2002, incorporated herein by reference.

“Plasticizer” refers to a compound capable of plasticizing or softening a polymer or a binder. Plasticizers can broaden the average molecular weight of a polymer in which they are included thereby lowering its glass transition temperature or softening point. Plasticizers also can reduce the viscosity of a polymer. The use of plasticizers is optional, but they can be included in a formulation to modify the properties and characteristics of the polymers used in the coat(s) or core of the formulation for convenient processing during manufacture of the coat(s) and/or the core of the formulation. Once the coat(s) and/or core has been manufactured, certain plasticizers can function to increase the hydrophilicity of the coat(s) and/or the core of the formulation in the environment of use. During manufacture of the coat(s) and/or core, the plasticizer can lower the melting temperature or glass transition temperature (softening point temperature) of the polymer or binder.

“Solid formulation” refers to a formulation that is neither liquid nor gaseous. Solid formulations include tablets, powders, microparticles, capsules, matrix forms, suppositories, sachets, troches, patches and lozenges. Solid formulations in the form of capsules contain a solid composition within a capsule that can be made of gelatin or other encapsulating material. Liquid formulations include liquid suspensions and elixirs.

“Swelling enhancer” refers to an excipient that swells rapidly resulting in an increase in the size of the tablet. At lower concentrations, these excipients can be used as super disintegrants; however at higher concentrations, e.g., at concentrations above about 5% w/w, these excipients function as swelling enhancers and increase the size of the matrix formulation.

### Treatment with Aspirin or Ketamine

It has been observed that COVID-19 infected patients suffer from both physical illness and mental stress. Conventional sedatives are mostly respiratory and cardiovascular depressants. Ketamine is observed to be able to enable sedation and anesthesia, attenuate depression and mitigate suicidal thoughts, without depressing respiratory or cardiovascular mechanics. It is contemplated that ketamine, an N-methyl-D-aspartate (NMDA) receptor antagonist, can effectively inhibit inflammation-induced neuropathy.

It has also been observed that aspirin can help reduce COVID-19 induced thrombosis and inflammation. Accordingly, aspirin alone or in combination with ketamine can be useful in treating and preventing symptoms of viral infections, in particular infections by coronaviruses.

One embodiment provides compositions and methods for treating or preventing the infection by a virus. The method, in some embodiments, entails administering a patient having, or at risk of developing, the infection an effective amount of aspirin, optionally in combination with ketamine. This can be a standalone therapy, or in combination with the other treatments disclosed herein.

Another embodiment provides a method of treating or preventing the infection by a virus, comprising administering a patient having, or at risk of developing, the infection an effective amount of ketamine. In some embodiments, the ketamine is orally administered. In some embodiments, a first portion of the ketamine disintegrates or dissolves intraorally, and a second portion of the ketamine is ingested and released in the gastrointestinal track of the patient.

In some embodiments, the patient suffers from a neuropathy. Non-limiting examples of the neuropathy include anosmia, cognitive or attention deficit, anxiety, depression, psychosis, seizure, suicidal behavior and combinations thereof.

The virus may be a coronavirus, such as SARS-CoV-2 or SARS-CoV. The virus may also be an influenza virus, such as the H5N1 influenza A virus or the H7N9 influenza A virus.

In some embodiments, the method entails administering to the subject a first amount of aspirin and a second amount of aspirin, wherein the first amount disintegrates or dissolves intraorally providing rapid release of the aspirin of the first amount, and the second amount is ingested and released in the gastrointestinal track of the subject.

In some embodiments, the method entails administering, or further administering, to the subject an effective amount of ketamine. In some embodiments, the administration is oral.

In some embodiments, the method entails administering, or further administering, to the subject a first amount of ketamine and a second amount of ketamine, wherein the first amount disintegrates or dissolves intraorally providing rapid release of the aspirin of the first amount, and the second amount is ingested and released in the gastrointestinal track of the subject.

In some embodiments, the ketamine is administered once, twice, three times or four times a day. In some embodiments, the last (or second, third) dose of ketamine is higher than the first dose.

In some embodiments, the administration of aspirin and/or ketamine starts immediately after diagnosis of the disease or condition, and preferably before the onset of the cytokine storm. In some embodiments, the first administration starts within 24 hours after diagnosis of the disease or condition. In some embodiments, the first administration starts within 1 day, 2 days, 3 days, 4 days, 5 days, 6 days or 7 days after diagnosis of the disease or condition. In some embodiments, the first administration is made before detected onset of a cytokine storm (or “cytokine release syndrome”).

Compositions to faciliate such administrations are also provided. One embodiment of the present disclosure provides a pharmaceutical composition comprising a first portion comprising a first amount of aspirin, and a second portion comprising a second amount of aspirin, wherein, upon oral administration to a subject, the first portion disintegrates or dissolves intraorally providing rapid release of the aspirin of the first portion, and the second portion is substantially more difficult than the first portion to disintegrate or dissolve intraorally but is ingestible and releasable in the gastrointestinal track of the subject.

In one aspect, the aspirin of the first portion is at a subtherapeutic amount, such as but not limited to, from about 10 mg to about 1000 mg. In one aspect, the amount of aspirin of the first portion is at least about 10 mg, or least about 20 mg, 30 mg, 40 mg, 50 mg, or 100 mg. In another aspect, the amount of aspirin of the first portion is no more than about 150 mg, 200 mg, 250 mg, 300 mg, 325 mg, 400 mg, 500 mg, 600 mg or 650 mg. In one aspect, the aspirin in the second portion is at a subtherapeutic amount, such as but not limited to, from about 10 mg to about 1000 mg. In one aspect, the amount of aspirin in the second portion is at least about 10 mg, or least about 20 mg, 30 mg, 40 mg, 50 mg, or 100 mg. In another aspect, the amount of aspirin in the second portion is no more than about 150 mg, 200 mg, 250 mg, 300 mg, 325 mg, 400 mg, 500 mg, 600 mg or 650 mg.

In one aspect, the aspirin of the first portion is at least about 10%, or 20%, or 30%, or 40%, of 50%, or 60%, or 70%, or 80%, or 80% of a therapeutically effective amount. In one aspect, the aspirin of the first portion is at most about 10%, or 20%, or 30%, or 40%, of 50%, or 60%, or 70%, or 80%, or 80% of a therapeutically effective amount. In one aspect, the aspirin of the second portion is at least about 10%, or 20%, or 30%, or 40%, of 50%, or 60%, or 70%, or 80%, or 80% of a therapeutically effective amount. In one aspect, the aspirin of the second portion is at most about 10%, or 20%, or 30%, or 40%, of 50%, or 60%, or 70%, or 80%, or 80% of a therapeutically effective amount.

In one aspect, the first portion of aspirin constitutes at least about 10% of the total aspirin. Alternatively, the first portion of aspirin constitutes at least about 20%, or 30%, or 40%, or 50%, or 60%, or 70%, or 80%, or 90% of the total aspirin. In some aspects, however, the first portion of aspirin can be less than about 20%, or 30%, or 40%, or 50%, or 60%, or 70%, or 80%, or 90% of the total aspirin. In a particular aspect, the first portion constitutes from about 40% to about 60%, or alternatively from about 45% to about 55% of the total aspirin.

In one aspect, the total amount of aspirin in the composition is less than about 50 mg, or 60 mg, or 70 mg, or 80 mg, or 90 mg, or 100 mg, or 120 mg, or 140 mg, or 150 mg, or 160 mg, or 165 mg, or 170 mg, or 180 mg, or 190 mg, or 200 mg. In another aspect, the total amount of aspirin in the composition is greater than about 150 mg, 160 mg, or 165 mg, or 170 mg, or 180 mg, or 190 mg, or 200 mg, or 250 mg, or 300 mg, or 400 mg, or 500 mg, or 600 mg, or 700 mg, or 800 mg, or 900 mg, or 1000 mg.

In one aspect, the total aspirin in the composition is at least about 81 mg. In one aspect, the first portion contains at least about 40 mg aspirin and the second portion contains at least about 40 mg aspirin. In some aspects, the second portion also contains at least about 500 mg niacin. In another aspect, the total aspirin in the composition is at least about 162 mg. In one aspect, the first portion contains at least about 81 mg aspirin and the second portion contains at least about 81 mg aspirin. In some aspects, the second portion also contains at least about 1000 mg niacin.

In one aspect, the total aspirin in the composition is at least about 121 mg. In one aspect, the first portion contains at least about 60 mg aspirin and the second portion contains at least about 60 mg aspirin. In some aspects, the second portion also contains at least about 500 mg niacin. In another aspect, the total aspirin in the composition is at least about 242 mg. In one aspect, the first portion contains at least about 121 mg aspirin and the second portion contains at least about 121 mg aspirin. In some aspects, the second portion also contains at least about 1000 mg niacin.

In one aspect, the total aspirin in the composition is at least about 203 mg. In one aspect, the first portion contains at least about 101 mg aspirin and the second portion contains at least about 101 mg aspirin. In some aspects, the second portion also contains at least about 500 mg niacin. In another aspect, the total aspirin in the composition is at least about 406 mg. In one aspect, the first portion contains at least about 203 mg aspirin and the second portion contains at least about 203 mg aspirin. In some aspects, the second portion also contains at least about 1000 mg niacin.

One embodiment of the present disclosure provides a pharmaceutical composition comprising a first portion comprising a first amount of ketamine, and a second portion comprising a second amount of ketamine, wherein, upon oral administration to a subject, the first portion disintegrates or dissolves intraorally providing rapid release of the ketamine of the first portion, and the second portion is substantially more difficult than the first portion to disintegrate or dissolve intraorally but is ingestible and releasable in the gastrointestinal track of the subject.

In one aspect, the ketamine of the first portion is at a subtherapeutic amount, such as but not limited to, from about 10 mg to about 1000 mg. In one aspect, the amount of ketamine of the first portion is at least about 10 mg, or least about 20 mg, 30 mg, 40 mg, 50 mg, or 100 mg. In another aspect, the amount of ketamine of the first portion is no more than about 150 mg, 200 mg, 250 mg, 300 mg, 325 mg, 400 mg, 500 mg, 600 mg or 650 mg. In one aspect, the ketamine in the second portion is at a subtherapeutic amount, such as but not limited to, from about 10 mg to about 1000 mg. In one aspect, the amount of ketamine in the second portion is at least about 10 mg, or least about 20 mg, 30 mg, 40 mg, 50 mg, or 100 mg. In another aspect, the amount of ketamine in the second portion is no more than about 150 mg, 200 mg, 250 mg, 300 mg, 325 mg, 400 mg, 500 mg, 600 mg or 650 mg.

In one aspect, the ketamine of the first portion is at least about 10%, or 20%, or 30%, or 40%, of 50%, or 60%, or 70%, or 80%, or 80% of a therapeutically effective amount. In one aspect, the ketamine of the first portion is at most about 10%, or 20%, or 30%, or 40%, of 50%, or 60%, or 70%, or 80%, or 80% of a therapeutically effective amount. In one aspect, the ketamine of the second portion is at least about 10%, or 20%, or 30%, or 40%, of 50%, or 60%, or 70%, or 80%, or 80% of a therapeutically effective amount. In one aspect, the ketamine of the second portion is at most about 10%, or 20%, or 30%, or 40%, of 50%, or 60%, or 70%, or 80%, or 80% of a therapeutically effective amount.

In one aspect, the first portion of ketamine constitutes at least about 10% of the total ketamine. Alternatively, the first portion of ketamine constitutes at least about 20%, or 30%, or 40%, or 50%, or 60%, or 70%, or 80%, or 90% of the total ketamine. In some aspects, however, the first portion of ketamine can be less than about 20%, or 30%, or 40%, or 50%, or 60%, or 70%, or 80%, or 90% of the total ketamine. In a particular aspect, the first portion constitutes from about 40% to about 60%, or alternatively from about 45% to about 55% of the total ketamine.

In one aspect, the total amount of ketamine in the composition is less than about 50 mg, or 60 mg, or 70 mg, or 80 mg, or 90 mg, or 100 mg, or 120 mg, or 140 mg, or 150 mg, or 160 mg, or 165 mg, or 170 mg, or 180 mg, or 190 mg, or 200 mg. In another aspect, the total amount of ketamine in the composition is greater than about 150 mg, 160 mg, or 165 mg, or 170 m g, or 180 mg, or 190 mg, or 200 mg, or 250 mg, or 300 mg, or 400 mg, or 500 mg, or 600 mg, or 700 mg, or 800 mg, or 900 mg, or 1000 mg.

One embodiment of the present disclosure provides a pharmaceutical composition comprising a first portion comprising a first amount of aspirin, and a second portion comprising a second amount of aspirin and also ketamine, wherein, upon oral administration to a subject, the first portion disintegrates or dissolves intraorally providing rapid release of the ketamine of the first portion, and the second portion is substantially more difficult than the first portion to disintegrate or dissolve intraorally but is ingestible and releasable in the gastrointestinal track of the subject.

Another embodiment of the present disclosure provides a pharmaceutical composition comprising a first portion comprising a first amount of aspirin and a first amount of ketamine, and a second portion comprising a second amount of aspirin and a second amount of ketamine, wherein, upon oral administration to a subject, the first portion disintegrates or dissolves intraorally providing rapid release of the ketamine of the first portion, and the second portion is substantially more difficult than the first portion to disintegrate or dissolve intraorally but is ingestible and releasable in the gastrointestinal track of the subject.

Methods of preparing a composition suitable for intraoral release are known in the art. In one aspect, the first portion further includes a film-coating agent, an excipient, a binder, a lubricant, or a plasticizer.

In one aspect, the first portion disintegrates or dissolves intraorally within about 10 minutes. In other aspects, the first portion disintegrates or dissolves intraorally within about 9 minutes, or about 8, or about 7, or about 6, or about 5, or about 4, or about 3 or about 2 minutes, or alternatively about 60 seconds, or about 50, or about 40, or about 30, or about 20, or about 10, or about 5 seconds.

In some aspects, the first portion is chewable. In some aspects, the first portion is in the form of molded triturate.

In one aspect, the first portion further includes an agent that promotes the oral or buccal absorption of aspirin. Non-limiting examples of such agents include bile acid salts, sodium lauryl sulfate, lysalbinic acid, salicylic acid, 5-methoxy salicylic acid, 3,4-dihydroxy phenyl acetic acid (DOPAC) and homovanillic acid and their sodium salts thereof. Other hydroxyaryl acids, such as 1-hydroxy-2-naphthoic acid, naphthoresorcyclic acid, ferulic acid, caffeic acid, resorcylic acid and gentisic acid, have similar effects.

The amount of hydroxyaryl or hydroxyaralkyl acid or salt, amide or ester derivatives thereof forms may vary over a wide range; in general, the identity and the amount of the hydroxyaryl or hydroxyaralkyl acids or salt, amide or ester thereof is used in connection with the drug in order to be effective in enhancing the absorption rate of the drug into the bloodstream.

In another aspect, the first portion further includes a disintegrant. Non-limiting examples of disintegrants include crospovidone, crystalline cellulose, hydroxypropylcellulose with a low degree of substitution, croscarmellose sodium, carmellose calcium, carboxystarch sodium, carboxymethyl starch sodium, potato starch, wheat starch, corn starch, rice starch, partly pregelatinized starch, and hydroxypropyl starch. One or two or more of these can be used together. Coating with a disintegrant also contributes to improvement of compression moldability.

The second and third portions of the composition can be prepared with methods known in the art for a typical oral dosage form suitable for GI absorption. Like the first portion, the second portion can also include a film-coating agent, an excipient, a binder, a lubricant, or a plasticizer.

Compared to the first portion, the second is substantially more difficult to disintegrate or dissolve intraorally. This can be achieved chemically or physically. For instance, the second portion can be physically harder. In one aspect, the second portion is compressed. In another aspect, the second portion has a hardness that is at least about 10 kilopascal (kp), or alternatively about 11, or 12, or 13, or 14, or 15, or 20, or 25 or 30 or 40 or 50 kp.

Hardness can be assessed by means commonly used in the art, for example, using commercially available hardness testers that are routinely used for assessing the hardness of pharmaceutical dosage forms.

In some aspects, the second portion further comprises a pharmaceutically acceptable flavoring agent not present in the first portion. The flavoring agent provides a flavor that alerts the patients that this portion should not be chewed and needs to be swallowed so as to increase patient compliance.

In one aspect, the aspirin in the second portion constitutes at least about 10% of the total aspirin. Alternatively, the aspirin in the second portion constitutes at least about 20%, or 30%, or 40%, or 50%, or 60%, or 70%, or 80%, or 90% of the total aspirin. In some aspects, however, the aspirin in the second portion can be less than about 20%, or 30%, or 40%, or 50%, or 60%, or 70%, or 80%, or 90% of the total aspirin. In a particular aspect, the aspirin in second portion constitutes from about 40% to about 60%, or alternatively from about 45% to about 55% of the total aspirin. In one aspect, the ratio of aspirin between the first portion and the second portion is about 1:1. Alternatively, the ratio is at least about 1:4, or 1:3, or 1:2 or 1:1.5, or is no more than about 4:1, 3:1, 2:1 or 1.5:1.

In one aspect, the ketamine in the second portion constitutes at least about 10% of the total ketamine. Alternatively, the ketamine in the second portion constitutes at least about 20%, or 30%, or 40%, or 50%, or 60%, or 70%, or 80%, or 90% of the total ketamine. In some aspects, however, the ketamine in the second portion can be less than about 20%, or 30%, or 40%, or 50%, or 60%, or 70%, or 80%, or 90% of the total ketamine. In a particular aspect, the ketamine in second portion constitutes from about 40% to about 60%, or alternatively from about 45% to about 55% of the total ketamine. In one aspect, the ratio of ketamine between the first portion and the second portion is about 1:1. Alternatively, the ratio is at least about 1:4, or 1:3, or 1:2 or 1:1.5, or is no more than about 4:1, 3:1, 2:1 or 1.5:1.

The pharmaceutical composition of the present disclosure can be in the form of a tablet or capsule. When in the form of a tablet, the second portion, in one aspect, is enclosed within the first portion or alternatively partially exposed.

When the composition is in the form of a tablet, the tablet can include an outer portion and an inter portion, with the outer portion containing the first portion and the inner portion containing the second portion and optionally the third portion.

In one aspect, the outer portion is formulated to dissolve in the oral cavity of a subject and to release the aspirin in the first portion across the oral mucosa of the subject. In one aspect, the inner portion is harder than the outer portion and is formulated for dissolving in stomach, intestines, or further distal in the gastrointestinal tract of the subject.

In one aspect, the inner portion comprises a texture on the surface that is recognizable by the tongue of a subject. In another aspect, the outer portion comprises a water soluble sugar or sugar substitute. In another aspect, the outer portion is surrounded by a thin shell to allow encapsulation of liquid, powder or gel in the outer portion.

In one aspect, the outer potion is flavored or sweetened. In one aspect, the tablet further comprises an intermediate layer between the outer and inner portions. In one aspect, the intermediate layer comprises enteric coating. In one aspect, the inner portion is formulated to absorb a biting shock and not break a tooth. In another aspect, the tablet comprises a layer of aspirin which breaks down in the mouth, but this layer has particles within it that don't completely break down in the mouth and stay full particles, such that there is partial intraoral release and, when the particles as swallowed, partial gastrointestinal release.

The pharmaceutical composition of the above embodiments can further include a third portion that comprises an effective amount of niacin. In one aspect, the third portion is in the form of controlled release. In another aspect, the third portion further comprises enteric coating. In yet another aspect, the third portion is enclosed in the first portion or the second portion.

### Treatment with Immune Checkpoint Inhibitors

Analysis of patients suffering from infections by SARS-CoV-2 shows that a majority of them developed lymphopenia (reduced levels of lymphocytes, in particular CD8+ T cells), leading to compromised immune response to the injection. Lymphopenia was also associated with high mortality of patient infected with SARS-CoV-2, as well as other viruses such as H5N1. It is contemplated herein that administration of a checkpoint inhibitor can rescue the compromised immune system, leading to boosted immune response to the infection, leading to recovery.

A checkpoint inhibitor targets immune checkpoints, which are key regulators of the immune system that, when stimulated, can dampen the immune response to an immunologic stimulus. Checkpoint therapy can block inhibitory checkpoints, strengthening immune system function.

Approved checkpoint inhibitors target the molecules CTLA4, PD-1, and PD-L1. PD-1 is the transmembrane programmed cell death 1 protein (also called PDCD1 and CD279), which interacts with PD-L1 (PD-1 ligand 1, or CD274). PD-L1 on the cell surface binds to PD1 on an immune cell surface, which inhibits immune cell activity. Among PD-L1 functions is a key regulatory role on T cell activities. It appears that (cancer-mediated) upregulation of PD-L1 on the cell surface may inhibit T cells that might otherwise attack. Antibodies that bind to either PD-1 or PD-L1 and therefore block the interaction may allow the T-cells to attack the tumor.

The first checkpoint antibody approved by the FDA was ipilimumab, approved in 2011 for treatment of melanoma. It blocks the immune checkpoint molecule CTLA-4.

The IgG4 PD1 antibody Nivolumab was approved in 2014. Nivolumab is approved to treat melanoma, lung cancer, kidney cancer, bladder cancer, head and neck cancer, and Hodgkin's lymphoma. Pembrolizumab (brand name Keytruda) is another PD1 inhibitor that was approved by the FDA in 2014. Keytruda is approved to treat melanoma and lung cancer.

Spartalizumab (PDR001) is a PD-1 inhibitor currently being developed by Novartis to treat both solid tumors and lymphomas.

In 2016, PD-L1 inhibitor atezolizumab was approved for treating bladder cancer.

In some embodiments, the checkpoint inhibitor is an inhibitor of CTLA-4. In some embodiments, the checkpoint inhibitor is an inhibitor of PD-1. In some embodiments, the checkpoint inhibitor is an inhibitor of PD-L1. In some embodiments, the inhibitor is a monoclonal antibody.

In accordance with one embodiment of the present disclosure, provided is a method of treating or preventing the infection by a virus. The method, in some embodiments, entails administering a patient having, or at risk of developing, the infection an effective amount of a checkpoint inhibitor.

The virus may be a coronavirus, such as SARS-CoV-2 or SARS-CoV. The virus may also be an influenza virus, such as the H5N1 influenza A virus or the H7N9 influenza A virus.

In some embodiments, the patient has developed or is at risk of developing lymphopenia following the infection. In some embodiments, the patient has not developed a cytokine release syndrome (cytokine storm). In some embodiments, the administration is before the patient potentially develops the cytokine storm.

### Treatment Methods and Uses

“Treatment” or “treating” is an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

“Prevention” or “preventing” means any treatment of a disease or condition that causes the clinical symptoms of the disease or condition not to develop. Compounds may, in some embodiments, be administered to a subject (including a human) who is at risk or has a family history of the disease or condition.

“Subject” refers to an animal, such as a mammal (including a human), that has been or will be the object of treatment, observation or experiment. The methods described herein may be useful in human therapy and/or veterinary applications. In some embodiments, the subject is a mammal. In one embodiment, the subject is a human.

The term “therapeutically effective amount” or “effective amount” of a compound described herein or a pharmaceutically acceptable salt, tautomer, stereoisomer, mixture of stereoisomers, prodrug, or deuterated analog thereof means an amount sufficient to effect treatment when administered to a subject, to provide a therapeutic benefit such as amelioration of symptoms or slowing of disease progression. For example, a therapeutically effective amount may be an amount sufficient to decrease a symptom of a disease or condition. The therapeutically effective amount may vary depending on the subject, and disease or condition being treated, the weight and age of the subject, the severity of the disease or condition, and the manner of administering, which can readily be determined by one or ordinary skill in the art.

The methods described herein may be applied to cell populations in vivo or ex vivo. “In vivo” means within a living individual, as within an animal or human. In this context, the methods described herein may be used therapeutically in an individual. “Ex vivo” means outside of a living individual. Examples of ex vivo cell populations include in vitro cell cultures and biological samples including fluid or tissue samples obtained from individuals. Such samples may be obtained by methods well known in the art. Exemplary biological fluid samples include blood, cerebrospinal fluid, urine, and saliva. In this context, the compounds and compositions described herein may be used for a variety of purposes, including therapeutic and experimental purposes. For example, the compounds and compositions described herein may be used ex vivo to determine the optimal schedule and/or dosing of administration of a compound of the present disclosure for a given indication, cell type, individual, and other parameters. Information gleaned from such use may be used for experimental purposes or in the clinic to set protocols for in vivo treatment. Other ex vivo uses for which the compounds and compositions described herein may be suited are described below or will become apparent to those skilled in the art. The selected compounds may be further characterized to examine the safety or tolerance dosage in human or non-human subjects. Such properties may be examined using commonly known methods to those skilled in the art.

### Combination Therapies

In one embodiment, the compounds disclosed herein may be used in combination with one or more additional therapeutic agent that are being used and/or developed to treat viral infections.

In some embodiments, the one or more additional therapeutic agent may be lopinavir and ritonavir, a drug combination approved to treat HIV, which optionally is used along with the flu drug oseltamivir (Tamiflu).

In some embodiments, the one or more additional therapeutic agent may be favipiravir, fingolimod, and/or methylprednisolone. In some embodiments, the one or more additional therapeutic agent may be bevacizumab.

In some embodiments, the one or more additional therapeutic agent may be chloroquine phosphate, chloroquine, or hydroxychloroquine sulfate.

In some embodiments, the one or more additional therapeutic agent may be remdesivir.

In some embodiments, the one or more additional therapeutic agent may be fumaric acid or a derivatives or prodrugs. “Fumaric acid” is the chemical compound with the formula HO₂CCH═CHCO₂H. The “salts and esters” of fumaric acid are known as fumarates, such as dimethyl fumarate (DMF) and monomethyl fumarate (MMF). Other examples include Fumaric acid esters (FAE), salts of monoethylfumarate (MEF) and Diroximel fumarate (DRF).

In some embodiments, the one or more additional therapeutic agent may be ketamine, niacin, aspirin or a combination thereof.

### Pharmaceutical Compositions and Modes of Administration

Compounds provided herein are usually administered in the form of pharmaceutical compositions. Thus, provided herein are also pharmaceutical compositions that contain one or more of the compounds described herein or a pharmaceutically acceptable salt, tautomer, stereoisomer, mixture of stereoisomers, prodrug, or deuterated analog thereof and one or more pharmaceutically acceptable vehicles selected from carriers, adjuvants and excipients. Suitable pharmaceutically acceptable vehicles may include, for example, inert solid diluents and fillers, diluents, including sterile aqueous solution and various organic solvents, permeation enhancers, solubilizers and adjuvants. Such compositions are prepared in a manner well known in the pharmaceutical art. See, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

The pharmaceutical compositions may be administered in either single or multiple doses. The pharmaceutical composition may be administered by various methods including, for example, rectal, buccal, intranasal and transdermal routes. In certain embodiments, the pharmaceutical composition may be administered by intra-arterial injection, intravenously, intraperitoneally, parenterally, intramuscularly, subcutaneously, orally, topically, or as an inhalant.

One mode for administration is parenteral, for example, by injection. The forms in which the pharmaceutical compositions described herein may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Oral administration may be another route for administration of the compounds described herein. Administration may be via, for example, capsule or enteric coated tablets. In making the pharmaceutical compositions that include at least one compound described herein or a pharmaceutically acceptable salt, tautomer, stereoisomer, mixture of stereoisomers, prodrug, or deuterated analog thereof, the active ingredient is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include lubricating agents such as talc, magnesium stearate, and mineral oil; wetting agents; emulsifying and suspending agents; preserving agents such as methyl and propylhydroxy-benzoates; sweetening agents; and flavoring agents.

The compositions that include at least one compound described herein or a pharmaceutically acceptable salt, tautomer, stereoisomer, mixture of stereoisomers, prodrug, or deuterated analog thereof can be formulated so as to provide quick, sustained or delayed release of the active ingredient after administration to the subject by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods disclosed herein employ transdermal delivery devices (“patches”). Such transdermal patches may be used to provide continuous or discontinuous infusion of the compounds described herein in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

For preparing solid compositions such as tablets, the principal active ingredient may be mixed with a pharmaceutical excipient to form a solid preformulation composition containing a homogeneous mixture of a compound described herein or a pharmaceutically acceptable salt, tautomer, stereoisomer, mixture of stereoisomers, prodrug, or deuterated analog thereof. When referring to these preformulation compositions as homogeneous, the active ingredient may be dispersed evenly throughout the composition so that the composition may be readily subdivided into equally effective unit dosage forms such as tablets, pills and capsules.

The tablets or pills of the compounds described herein may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation may include solutions and suspensions in pharmaceutically acceptable, aqueous or organic solvents, or mixtures thereof, and powders. The liquid or solid compositions may contain suitable pharmaceutically acceptable excipients as described herein. In some embodiments, the compositions are administered by the oral or nasal respiratory route for local or systemic effect. In other embodiments, compositions in pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

### Dosing

The specific dose level of a compound of the present application for any particular subject will depend upon a variety of factors including the activity of the specific compound employed, the age, body weight, general health, sex, diet, time of administration, route of administration, and rate of excretion, drug combination and the severity of the particular disease in the subject undergoing therapy. For example, a dosage may be expressed as a number of milligrams of a compound described herein per kilogram of the subject's body weight (mg/kg). Dosages of between about 0.1 and 150 mg/kg may be appropriate. In some embodiments, about 0.1 and 100 mg/kg may be appropriate. In other embodiments a dosage of between 0.5 and 60 mg/kg may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject.

The daily dosage may also be described as a total amount of a compound described herein administered per dose or per day. Daily dosage of a compound of Formula I may be between about 1 mg and 4,000 mg, between about 2,000 to 4,000 mg/day, between about 1 to 2,000 mg/day, between about 1 to 1,000 mg/day, between about 10 to 500 mg/day, between about 20 to 500 mg/day, between about 50 to 300 mg/day, between about 75 to 200 mg/day, or between about 15 to 150 mg/day.

When administered orally, the total daily dosage for a human subject may be between 1 mg and 1,000 mg, between about 1,000-2,000 mg/day, between about 10-500 mg/day, between about 50-300 mg/day, between about 75-200 mg/day, or between about 100-150 mg/day.

The compounds of the present application or the compositions thereof may be administered once, twice, three, or four times daily, using any suitable mode described above. Also, administration or treatment with the compounds may be continued for a number of days; for example, commonly treatment would continue for at least 7 days, 14 days, or 28 days, for one cycle of treatment. Treatment cycles are well known in cancer chemotherapy, and are frequently alternated with resting periods of about 1 to 28 days, commonly about 7 days or about 14 days, between cycles. The treatment cycles, in other embodiments, may also be continuous.

In a particular embodiment, the method comprises administering to the subject an initial daily dose of about 1 to 800 mg of a compound described herein and increasing the dose by increments until clinical efficacy is achieved. Increments of about 5, 10, 25, 50, or 100 mg can be used to increase the dose. The dosage can be increased daily, every other day, twice per week, or once per week.

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. A method of treating or preventing the infection by a virus, comprising administering a patient having, or at risk of developing, the infection an effective amount of ketamine.

2. The method of claim 1, wherein the ketamine is orally administered.

3. The method of claim 2, wherein a first portion of the ketamine disintegrates or dissolves intraorally, and a second portion of the ketamine is ingested and released in the gastrointestinal track of the patient.

4. The method of claim 1, further comprising administering to the patient an effective amount of aspirin.

5. The method of claim 4, wherein a first portion of the aspirin disintegrates or dissolves intraorally, and a second portion of the aspirin is ingested and released in the gastrointestinal track of the patient.

6. A method of treating or preventing the infection by a virus in a subject, comprising orally administering to the subject a first amount of aspirin and a second amount of aspirin, wherein the first amount disintegrates or dissolves intraorally providing rapid release of the aspirin of the first amount, and the second amount is ingested and released in the gastrointestinal track of the subject.

7. The method of claim 6, wherein the second amount of aspirin is administered with ketamine.

8. The method of claim 6, wherein the first amount of aspirin is administered with ketamine.

9. The method of claim 1, wherein the virus is a coronavirus.

10. The method of claim 9, wherein the coronavirus is SARS-CoV-2 or SARS-CoV.

11. The method of claim 1, wherein the virus is an influenza virus.

12. The method of claim 11, wherein the influenza virus is the H5N1 influenza A virus or the H7N9 influenza A virus.

13. The method claim 1, wherein the patient suffers from a neuropathy.

14. The method of claim 13, wherein the neuropathy is selected from the group consisting of anosmia, cognitive or attention deficit, anxiety, depression, psychosis, seizure, suicidal behavior and combinations thereof.
